(19) United States
(12) Patent Application Publication
(10) Pub. No.: US 2025/0229019 A1
(43) Pub. Date: Jul. 17, 2025

(54) PUMP WITH EVACUATED DRIVER

(71) Applicant: AG IP HOLDING, LLC, Okemos, MI (US)

(72) Inventor: Patrick Kenneth Powell, Grosse Pointe Park, MI (US)

(21) Appl. No.: 18/854,707

(22) PCT Filed: Apr. 11, 2023
(86) PCT No.: PCT/US2023/018117

§ 371 Date: Oct. 7, 2024

Related U.S. Application Data
(60) Provisional application No. 63/329,627, filed on Apr. 11, 2022.
(60) Provisional application No. 63/342,653, filed on May 17, 2022.
(60) Provisional application No. 63/345,930, filed on May 26, 2022.
(60) Provisional application No. 63/358,334, filed on Jul. 5, 2022.
(60) Provisional application No. 63/358,337, filed on Jul. 5, 2022.
(60) Provisional application No. 63/359,262, filed on Jul. 8, 2022.
(60) Provisional application No. 63/388,405, filed on Jul. 12, 2022.
(60) Provisional application No. 63/389,205, filed on Jul. 14, 2022.
(60) Provisional application No. 63/389,496, filed on Jul. 15, 2022.
(60) Provisional application No. 63/405,173, filed on Sep. 9, 2022.
(60) Provisional application No. 63/416,647, filed on Oct. 17, 2022.
(60) Provisional application No. 63/482,116, filed on Jan. 30, 2023.

(51) Int. Cl. A61M 5/145; F04B 9/12

(52) U.S. Cl. CPC A61M 5/1454; F04B 9/1215

(57) ABSTRACT

A pump includes a container that has an interior volume for holding a substance. There is at least one outlet that is associated with the interior volume for discharging the substance. A driver is disposed in the interior volume. The driver has a stored potential energy that is releasable as kinetic energy and, upon release, the driver expands against the substance in the interior volume and thereby discharge the substance through the at least one outlet.

### BACKGROUND

A medical pump is a type of pump used in healthcare to deliver medicaments to patients. The pumps are used in a variety of settings including hospitals, clinics, and the home, with the latter requiring higher levels of self-administration by the patient. The pump typically consists of a container, an inlet for filling the container, an outlet for divesting the container contents, an actuator for expelling the contents, along with a variety of needles, nozzles, and tubing fitted to direct the contents between the container and the patient.

One type of pump used in medicine is a syringe, which typically includes a body, a plunger, and a push-pull stem. The syringe is ubiquitous in medicine for administration of medicaments, and is also used in other fields to withdraw, measure, and distribute substances.

Despite widespread use, the syringe has unaddressed difficulties that are especially acute for self-administration by the patient including techniques of accurately connecting with the body, ergonomically moving the plunger, problems with dosing accuracy, maintaining sterility in the home environment, medicament preservation of active pharmaceutical ingredients and active substance expiration including cold chain, oxygen (air) exposure and light controls. Safety with sharps handling and disposal is an additional challenge especially for self-administration accessibility. Other challenges include the delivery of higher viscosity fluids in larger volumes, such as with biologics, which often require extensive delivery times for safe and effective treatment. The issues create further challenges when adopting pumps for infusion and wearables, including higher complexity which leads to administration errors and patient non-compliance with treatment regimens, resulting in substandard therapy.

The field of drug therapy self-administration offers patients additional value including convenience of home therapy, less travel and time to visit clinics, less burden on care providers, less exposure to pathogens in clinic settings, more personal privacy, lower overall personal and system costs, and greater self-determination in controlling therapy results. Self-administration is expected to lead to faster recovery, improved satisfaction, and patient independence all resulting in higher quality of life.

### SUMMARY

A pump according to an example of the present disclosure includes a container that has an interior volume for holding a substance, at least one outlet associated with the interior volume for discharging the substance, and a driver disposed in the interior volume. The driver has a stored potential energy that is releasable as kinetic energy and, upon release, the driver is expandable against the substance in the interior volume to thereby discharge the substance through the at least one outlet.

In a further embodiment of the foregoing embodiment, the driver includes a casing and a spring device in the casing. The casing is evacuated to define a pressure differential that stresses the spring device to provide the stored potential energy.

In a further embodiment of any of the foregoing embodiments, the driver is configured to release the stored potential energy by release of the pressure differential.

In a further embodiment of any of the foregoing embodiments, the driver has a pressure equalization port that is operable to release the pressure differential and, upon release, the spring device expands to increase the volume of the casing and thereby generates a secondary vacuum in the casing.

In a further embodiment of any of the foregoing embodiments, the container includes first and second ends. The at least one outlet is in the first end, and the pressure equalization port is in the first end.

In a further embodiment of any of the foregoing embodiments, the driver includes first and second segments. The first segment includes a first seal that seals against an interior surface of the container and the second segment includes a second seal that seals against an interior surface of the container. The first and second seals isolate a region from the remainder of the interior volume.

In a further embodiment of any of the foregoing embodiments, the driver includes a spring device in the region. The region is evacuated to define a pressure differential that stresses the spring device to provide the stored potential energy. The first segment is translatable in the container, the second segment is fixed in the container, and upon release of the pressure differential the spring device moves the second segment against the substance to discharge the substance through the at least one outlet.

A further embodiment of any of the foregoing embodiments includes a conduit in the container and extends through the driver. The conduit connects the interior volume with the at least one outlet for discharging the substance through the conduit.

A further embodiment of any of the foregoing embodiments includes a passive valve associated with the conduit. The passive valve is held closed by the pressure differential and opening upon release of the pressure differential to permit the substance to flow into the conduit.

In a further embodiment of any of the foregoing embodiments, the at least one outlet includes an attachment selected from the group consisting of a needle, a roller ball, a brush, a foam applicator, a directional nozzle, and combinations thereof.

A further embodiment of any of the foregoing embodiments includes a stem that is attached with the driver. The stem is moveable to retract the driver in the container and thereby draw the substance into the interior volume.

In a further embodiment of any of the foregoing embodiments, the driver occupies a region in the container, and the region is fluidly isolated from the interior volume.

A further embodiment of any of the foregoing embodiments includes a thermal material in the driver. The thermal material is actively responsive to release of the driver to change the temperature of the substance.

In a further embodiment of any of the foregoing embodiments, the container includes a window and the driver includes a marker. The marker becomes visible through the window upon release of the driver.

A pump according to an example of the present disclosure includes a container that has an interior volume, a substance in the interior volume, at least one outlet associated with the interior volume for discharging the substance, and at least one evacuated driver disposed in the interior volume. The evacuated driver defines a pressure differential that stresses a spring device of the evacuated driver to store a potential energy. The evacuated driver has a pressure equalization port that is operable to release the pressure differential and, upon release, to cause the potential energy in the spring device to convert to kinetic energy such that the evacuated driver expands against the substance in the interior volume and thereby discharge the substance through the at least one outlet.

In a further embodiment of any of the foregoing embodiments, the pressure equalization port includes a regulator that restricts inflow into the evacuated driver in proportion to expansion of the evacuated driver.

In a further embodiment of any of the foregoing embodiments, the regulator includes at least one passive flow restrictor selected from an orifice, a flow restriction tube, an open cell foam, a viscous fluid, and combinations thereof.

In a further embodiment of any of the foregoing embodiments, the regulator includes at least one active flow control selected from a valve, an electrical element, a solenoid, a phase change material, a thermally degradable material, a fixed volume pump, and combinations thereof.

In a further embodiment of any of the foregoing embodiments, the regulator includes the fixed volume pump, the fixed volume pump being operable to release a fixed volume of air into the evacuated driver to incrementally release the pressure differential.

In a further embodiment of any of the foregoing embodiments, the at least one evacuated driver includes two evacuated drivers, and each of the two evacuated drivers is segmented into a moveable first segment, a fixed second segment, and the two evacuated drivers are interconnected by a shaft.

A method for controlling discharge from a pump according to an example of the present disclosure includes providing a pump as in any of the foregoing embodiments, connecting the pump to a patient for delivery of a medicament, releasing the pressure differential via the pressure equalization port to cause the potential energy in the spring device to convert to kinetic energy such that the evacuated driver expands against the medicament in the interior volume and thereby discharges the medicament through the at least one outlet. The expansion draws an inflow into the evacuated driver through the pressure equalization port, and after delivery of the medicament to the patient, disconnects the pump from the patient.

An example method for assembling a pump according to an example of the present disclosure includes providing a container that has an interior container volume and an outlet associated with the interior volume, introducing a medicament into the interior volume, installing an evacuated driver at least partially into the interior volume of the container. The evacuated driver has a stored potential energy that is releasable as kinetic energy and, upon release, the evacuated driver is expandable against the medicament in the interior volume to thereby discharge the substance through the outlet.

The present disclosure may include any one or more of the individual features disclosed above and/or below alone or in any combination thereof.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1 illustrates a pump with a substance driver.

FIG. 2 illustrates the pump of FIG. 1 during release of the driver to discharge the substance.

FIG. 3 illustrates another example of the pump with inlet and outlet regulators.

FIG. 4 illustrates a pump with a substance driver that has two segments.

FIG. 5 illustrates the pump of FIG. 4 during release of the driver to discharge the substance.

FIG. 6 illustrates another example of a pump adapted for a vial.

FIG. 7 illustrates another example pump with a stem for drawing the substance into the pump and for releasing the driver.

FIG. 8 illustrates the pump of FIG. 7 while drawing the substance into the pump.

FIG. 9 illustrates the pump of FIG. 7 during release of the driver to discharge the substance.

FIG. 10 illustrates an example in which multiple pumps are used.

FIG. 11 illustrates a pump with an orifice regulator.

FIG. 12 illustrates a pump with a regulator that has multiple, differently sized inlets.

FIG. 13 illustrates a pump with a fixed volume pump regulator.

FIG. 14 illustrates a pump with an elongated flow restriction tube regulator.

FIG. 15A illustrates a pump with an open cell foam regulator.

FIG. 15B illustrates the pump of FIG. 15A during release of the driver.

FIG. 16 illustrates a pump with a spring-loaded regulator.

FIG. 17 illustrates the pump of FIG. 17 during release of the driver.

FIG. 18 illustrates a pump with a closure member and electric circuit regulator.

FIG. 19 illustrates a pump with a viscous fluid regulator.

FIG. 20 illustrates a pump with a mechanical clock valve regulator.

FIG. 21 illustrates a pump with an electro-mechanical regulator.

FIG. 22 illustrates a pump with a needle outlet.

FIG. 23 illustrates a pump with a roller ball outlet.

FIG. 24 illustrates a pump with a brush outlet,

FIG. 25 illustrates a pump with a foam applicator tip outlet.

FIG. 26 illustrates a pump with a radial outlet,

FIG. 27 illustrates a pump that has two drives.

FIG. 28 illustrates the pump of FIG. 27 during release of one driver to draw substance into the pump.

FIG. 29 illustrates the pump of FIG. 28 during release of the second driver to discharge the substance.

FIG. 30 illustrates a pump with an asymmetrical driver.

FIG. 31 illustrates the pump of FIG. 30 during release of the driver.

FIG. 32 illustrates the pump of FIG. 30 upon full release of the driver.

In this disclosure, like reference numerals designate like elements where appropriate and reference numerals with the addition of one-hundred or multiples thereof designate modified elements that are understood to incorporate the same features and benefits of the corresponding elements.

### DETAILED DESCRIPTION

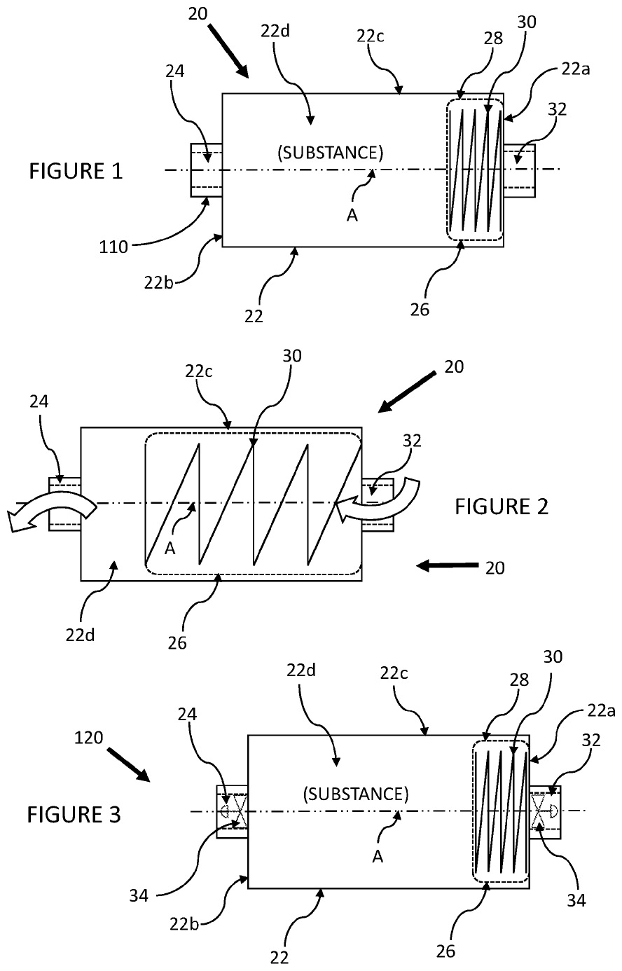

FIG. 1 schematically illustrates a pump 20. The pump 20 may be used for delivery of a fluid, such as but not limited to, a liquid, a gas, a semi-solid, a gel, a suspension, a flowable powder, a moveable solid, or combinations of these. A contemplated implementation of the pump 20 is in the medical industry for delivery of a medicament to a patient, although the pump 20 is not limited to such a use and may be used in other types of end-uses to move fluids. In some instances, the substance may be initially solid (as with a frozen vaccine or drug suspended in wax) and then softened for delivery, or the substance may be a mixture of other substances, such as a reconstitution with a fluid and a powder (e.g., a lyophilized pharmaceutical).

The pump 20 is generally comprised of a container 22. In this example, the container 22 defines a central axis A and has first and second end walls 22a/22b and a side wall or walls 22c. The walls 22a/22b/22c define an interior container volume 22d for holding a substance (fluid) that is to be pumped. As an example, the size of the interior container volume 22d may generally be from 1 mL to 1000 mL, but the size is not particularly limited and may be scaled up or down for a particular implementation. In this example, the container 22 is cylindrical and is formed of a plastic material. However, the geometry and material of the container 22 can be varied. For example, the container 22 may alternatively be made of metal, glass, ceramic, or elastomer, or combinations of different materials, including multilayered structures. The container 22 may be opaque, translucent, or transparent to visible light, and may be symmetrical or asymmetrical. The substance may be pre-loaded in the container 22 such that the pump 20 is ready for use, but the substance may alternatively be loaded into the container on-demand in preparation of the pump 20 for an imminent use.

The pump 20 includes at least one outlet 24 that is connected with the interior container volume 22a for discharging the substance. In the illustrated example, there is one outlet 24 and it is located at the second end wall 22b. It is to be appreciated, however, that there could be additional outlets, such as two outlets, three outlets, or more than three outlets, and the outlet or outlets may be located elsewhere on the container 22.

The pump 20 further includes a driver 26 disposed in the interior container volume 22d. The driver 26 driver occupies a region of the container 22, and the region is fluidly isolated from (i.e. sealed from) the interior container volume 22d. The driver 26 has a stored potential energy that is releasable as kinetic energy. Upon release, as shown in FIG. 2, the driver 26 expands along a linear axial movement direction in the container 22 to take up at least a portion of the interior container volume 22d. In doing so, the driver 22 applies pressure against the substance in the interior container volume 22d and thereby causes the substance to discharge from the container 22 through the outlet 24.

The driver 26 in this example includes a casing 28 and a spring device 30 in the casing 28. The casing 28 is expandable and collapsible. In this regard, the casing 28 may include, but is not limited to, an elastically flexible sack, a convoluted sack, or other mechanical structure that permits the casing to expand/collapse. For example, the casing 28 may be of a monolayer or multi-layer wall construction. The interior of the casing 28 is evacuated and thus defines a pressure differential across the spring device 30, relative to the surrounding ambient environment pressure. That is, the vacuum in the casing 28 relative to the surrounding ambient pressure causes the casing 28 to collapse around, and thus stress, the spring device 30 to provide the stored potential energy. In general, the vacuum pressure in the casing 28 may be a low as <10⁻³ Pa but may be scaled up or down for a particular implementation. The spring device 30 in the illustrated example is a coil spring. Alternatively, the spring device 30 may include, but is not limited to, a compressible foam, a compressible elastic, a compressible textile, a compressible fluid, a collapsible lattice structure, torsion spring, constant force spring (e.g., clock spring) or combinations of different types of these or other types of springs, as long as the spring device 30 can be stressed under the vacuum force of the collapsed casing 28 and to store potential energy and then elastically recover to release the potential energy once the pressure differential is equalized. In some instances, the spring device 30 may compress when stressed (e.g., a coil spring or foam), but in other instances the spring device 30 may twist or otherwise deform when stressed (e.g., torsion spring or a constant force spring).

The vacuum in the casing 28 holds the spring device 30 in its stressed, elevated potential energy state (relative to the spring device 30 at rest) until the pressure differential is released. The pressure differential is released by opening the casing 28 to the surrounding ambient environment. Once opened, air or other substances from the surrounding environment enters into the casing 28, thereby at least partially equalizing the initial vacuum in the casing 28 with the ambient surroundings. Once equalized, or as the vacuum equalizes, the vacuum force holding the casing 28 in its collapsed state decreases and the potential energy of the spring device 30 converts to kinetic energy. Under the force of the kinetic energy of the spring device 30, the casing 28 expands to take up at least a portion of the interior container volume 22d. The expansion of the casing 28 increases the volume of the casing 28, which generates a secondary vacuum in the casing 28 that draws an inflow of air or other substances from the surrounding environment into the casing 28 to equalize the secondary vacuum. In the figures herein, flow is represented by block arrows. The inflow to equalize this secondary vacuum may be controlled to thereby control the expansion on the casing 28 and concomitant discharge of the substance from the pump 20, as the secondary vacuum is generally proportional to the displaced interior container volume 22d drawing the ambient surroundings into the casing 28 through inlet port 32.

The casing 28 may be opened to release the pressure differential via an inlet port 32. The inlet port 32 extends thorough the first end wall 22a and connects the interior of the casing 28 to the surrounding ambient environment. The inlet port 32 is initially sealed, thereby preserving the vacuum in the casing 28 so that the potential energy is stored until released to activate the pump 20. The release can be triggered manually or in an automated fashion by opening the inlet port 32, such as by breaking a seal associated with the inlet port 32, opening a valve associated with the inlet port 32, or by operating or activating a regulator associated with the inlet port 32.

FIG. 3 illustrates another example of a pump 120 that is the same as the pump 20 but includes a regulator 34 for controlling the inflow to equalize the secondary vacuum. The flow restriction serves to control expansion of the driver 26, which in turn controls the discharge rate of the substance from the outlet 24. For example, relatively low restriction of the inflow results in a rapid pressure equalization, rapid expansion of the driver 26, and a concomitant high rate of discharge. Inversely, relatively high restriction of the inflow results in a slower, gradual pressure equalization, slower expansion of the driver 26, and a concomitant low rate of discharge. Thus, the regulator 34 may be tailored to control the rate of inflow and thereby control the discharge rate of the substance. The initial vacuum pressure in the driver 26, characteristics of the spring device 30, and volume of the casing 28 may also be selected in cooperation with the regulator 34 to modulate the discharge rate.

The regulator 34 is selected from an orifice, a flow restriction tube, a fixed volume pump, an open cell foam, a valve, an electrical element, a viscous fluid, a mechanical clock valve, a solenoid, a wax, a thermally degradable material, or combinations thereof, further embodiments of which will be discussed in subsequent examples. A regulator 34 such as an orifice, a flow restriction tube, an open cell foam, a viscous fluid, or combinations thereof operate passively, while other types of regulators, such as a valve, an electrical element, a solenoid, a phase change material, a thermally degradable material, a fixed volume pump, or combinations thereof, are operated with an active control to modulate flow restriction and thus discharge. For example, the vacuum created per displacement of the driver 26 decreases as the driver 26 expands (because the linear distance of the displacement creates a smaller change in volume). Thus, if left unmodulated, the discharge rate may vary as the driver 26 expands. In this regard, the active regulators may be used to facilitate leveling out the speed by which the substance is expelled from the container 22.

Additionally or alternatively, the discharge rate may be directly controlled by a regulator 34 associated with the outlet 24. For example, a valve, orifice, or other type of regulator 34 restricts outflow of the substance to control the discharge rate. In further examples, regulators 34 are used in association with both the inlet port 32 and the outlet 24. Such use at both the inlet 32 and outlet 24 may facilitate finer control over the discharge rate.

In one example, the regulator 34 varies the inflow in order to facilitate a more uniform discharge of the substance. For instance, for many types of springs the spring force decreases as the spring recovers (e.g., decompresses). Therefore, with no control of the inflow, the force of expansion against the substance would decrease over the time period in which the spring recovers. Since the force of expansion is proportional to the rate of discharge of the substance from the pump 20, the rate of discharge would also decrease over the time period in which the spring recovers. In this regard, the inflow can be increased over the time period in which the spring recovers in order to produce a constant force of expansion, and thus a constant rate of discharge.

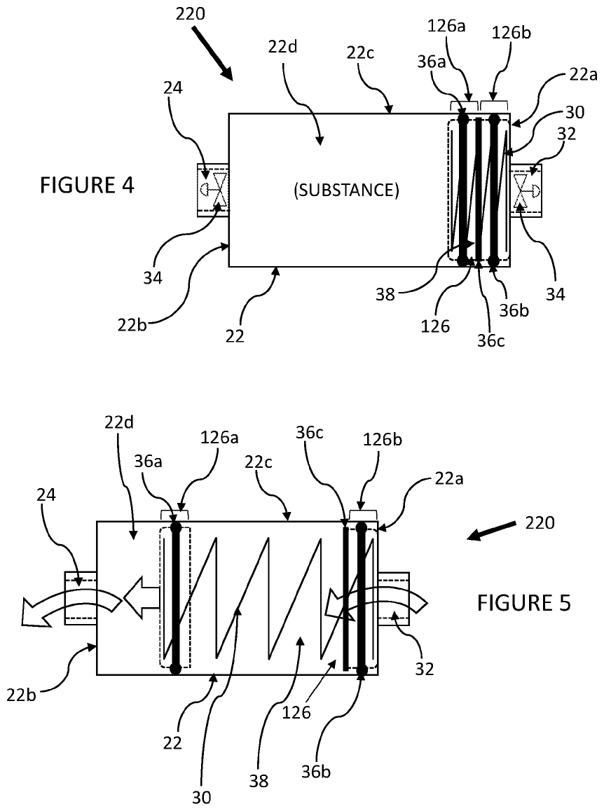

FIG. 4 illustrates another example pump 220 in which the driver 126 includes two segments 126a/126b. The first segment 126a is linearly translatable in the container 22 (in the left-right direction in the figure), while the second segment 126b is fixed in the container 22. In this example, the driver 126 excludes the afore-described casing 28. Instead, the first segment 126a includes a seal 36a that dynamically seals against the interior surface of the wall 22c of the container 22. Another seal 36c seals between the segments 126a/126b. The seals 36a/36b thus isolate a region 38 from the remainder of the interior container volume 22d and thus functions similarly to the casing 28 in that regard. The spring device 30 is located in the region 38, which is evacuated. The resulting pressure differential holds the first segment 126a in a retracted position in which it stresses the spring device 30 to store the potential energy. Upon release of the pressure differential as shown in FIG. 5, the spring device 30 is permitted to expand, pushing the first segment 126a against the substance (to the left in the figure) and thereby discharging the substance through the outlet 24. In the embodiments herein, the driver 26/126 may be pre-fabricated with a seal 36c between first segment 126a/126b to hold the spring (30) under a pressure differential and then assembled into the container 22 to provide the pumps disclosed herein.

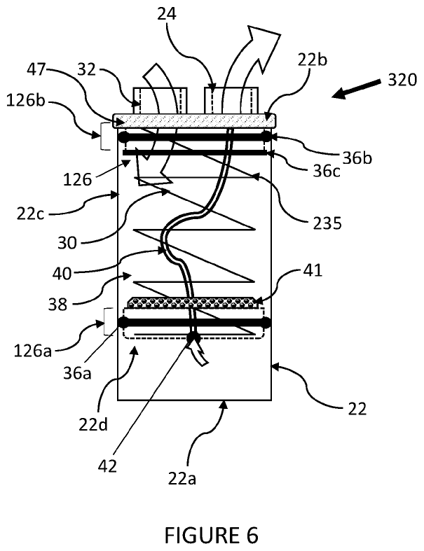

FIG. 6 illustrates another example pump 320, which is implemented as a self-pumping vial. The pump 320 is the same as the pump 220 except that container 22 is a vial and the inlet port 32 and the outlet 24 are both located at the second end wall 22b, which in this case may be a cap locked on the vial. The outlet 24 is connected with the interior container volume 22d via a conduit 40 that extends through the driver 126. The driver 126 is shown in its expanded, released state. Upon release of the pressure differential, the substance is discharged through the conduit 40 to the outlet 24. The vial is thus configured, upon release of the pressure differential, to self-pump the substance out of the vial. Such an implementation may be useful in a wearable medical product, as it eliminates the need for a user to transfer the substance into a syringe at the point of care. Vials may have various shapes and in that regard the driver 126 may be expandable in a direction away from the axis of pumping motion in order to fill the full inner diameter of the vial.

Optionally, the pump 320 may further include a thermal material 41 that serves to change the temperature of the substance. The thermal material 41 includes one or more compounds or elements that are either exothermically or endothermically reactive to warm or cool, respectfully, the substance. The warming may be used to warm drugs, biologics, blood, hydration, or plasma to mitigate thermal shock when entering the body, or to reduce a dynamic viscosity of a viscous fluid or solid to enable injection through an elongated needle. The cooling may be used to reduce injection pain at a needle injection site of a patient.

In one example, the thermal material 41 includes iron powder, water, a salt, and activated carbon or vermiculite. Upon inflow of air (oxygen) into the driver 126, the iron oxidizes in an exothermic reaction to produce heat that warms the substance. The water serves as a catalyst for the oxidation reaction, the salt regulates the reaction rate and prevents the mixture from rapidly drying out, and the activated carbon or vermiculite serves as an insulator to help retain the heat. In additional examples, the thermal material 41 includes one or more of calcium nitrate, magnesium sulfate, urea, calcium nitrate, potassium chloride, baking soda, salts of various forms and types, ammonium nitrate, silica, cellulose, glycol, sodium polyacrylate, and derivatives of these compounds.

The pump 320 may further include a passive valve 42 located at the inlet of the conduit 40. The passive valve 42 is initially held in a closed state by the vacuum in the region 38. Upon release of the vacuum, the passive valve 42 opens, permitting flow of the substance into the conduit 40. A pressurized gas pocket may initially be provided in the interior container volume 22d with the substance to facilitate keeping the passive valve 42 in the closed state initially. The passive valve 42 eliminates the need for a separate valve that has to be operated by the user or through a controller.

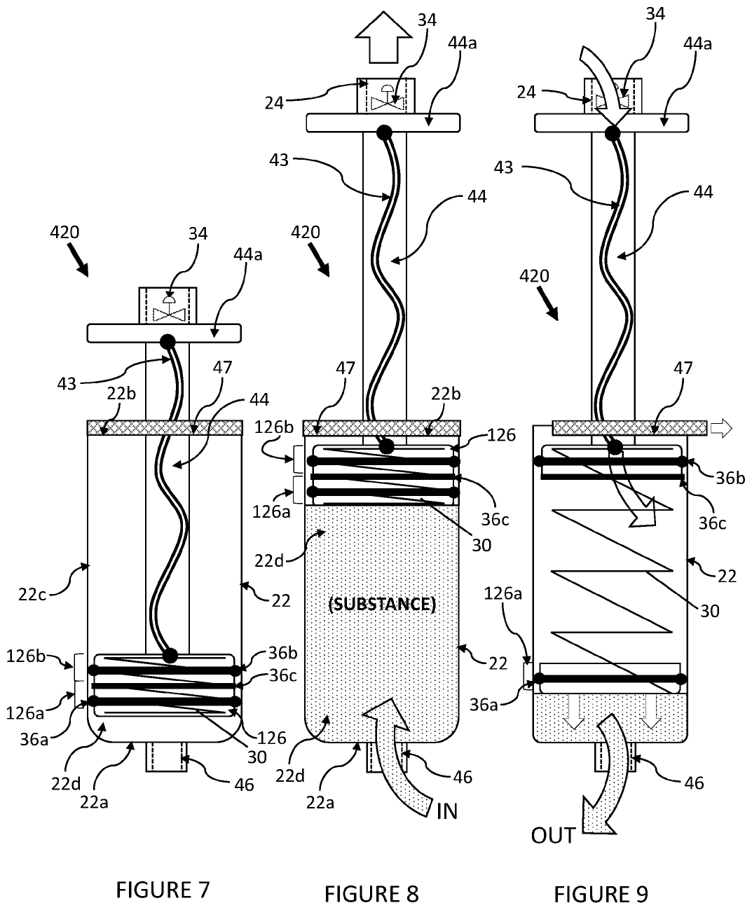

FIG. 7 illustrates another example pump 420, which is similar to the pump 320 but includes a stem 44 that is attached with the driver 126. A portion of the stem 44 projects from the container 22 and includes a handle 44a for operating the pump 420. The driver 126 is initially situated toward the first end wall 22a of the container 22. The stem 44 is moveable via the handle 44a to retract the driver 126 in the container 22 toward the second end wall 22b. As the driver 126 is retracted, a vacuum is created in the interior container volume 22d. The vacuum draws the substance through a port 46 into the interior container volume 22d.

Upon retraction as shown in FIG. 8, a lock 47 engages the stem 44 (see also FIG. 9) to the container 22 such that the second segment 126b of the driver 126 becomes fixed in position adjacent the second end wall 22b. Upon release of the pressure differential of the driver 126 as shown in FIG. 9, the pressure differential equalizes via a conduit 43 that extends through the stem 44 to the inlet 24. A regulator 34 as discussed above may be provided in association with the inlet 24 for controlling discharge. A user may activate the release from the stem 44 or handle 44a. The spring device 30 expands and pushes the first segment 126a against the substance to thereby discharge the substance through the port 46.

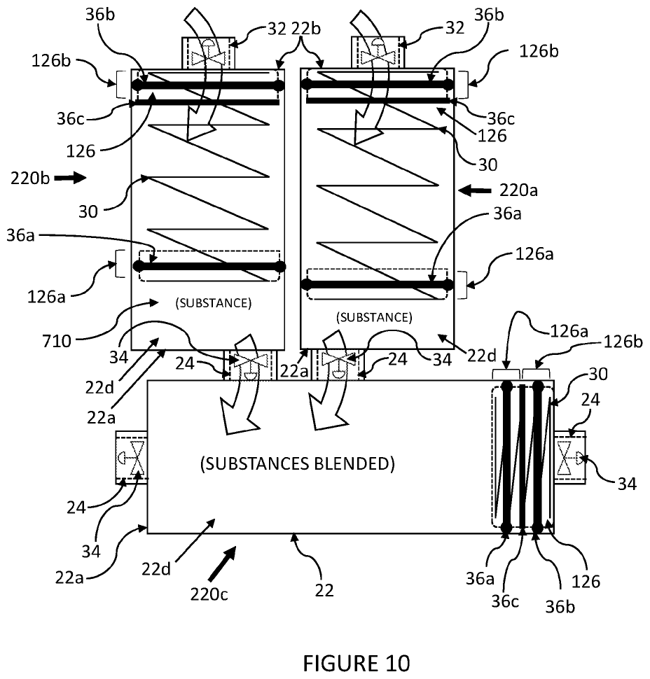

FIG. 10 illustrates an additional example in which multiple pumps 220a/220b/220c are combined. Each of the pumps 220a/220b/220c are similar to the pump 220 described previously. The pumps 220a/220b are configured as supply pumps and the pump 220c is configured as a blend pump. In this regard, the outlets 24 of supply pumps 220a/220b are connected to the interior container volume 22d of the blend pump 220c. The supply pumps 220a/220b contain different substances and, upon release of the drivers 126 of the supply pumps 220a/220b, the substances are discharged into the blend pump 220c where the substances mix to provide a blended substance. The driver 126 of the blend pump 220c can thereafter be released to deliver the mixture from the outlet 24 of the blend pump 220c. A timing differential may be deployed between injections of the substances between pump 220a, 220b, and 220c. The timing enables the proper mixing of substances. For example, the reconstitution of a lyophilized powdered drug in 220c may require time to enable full rehydration prior to an injection.

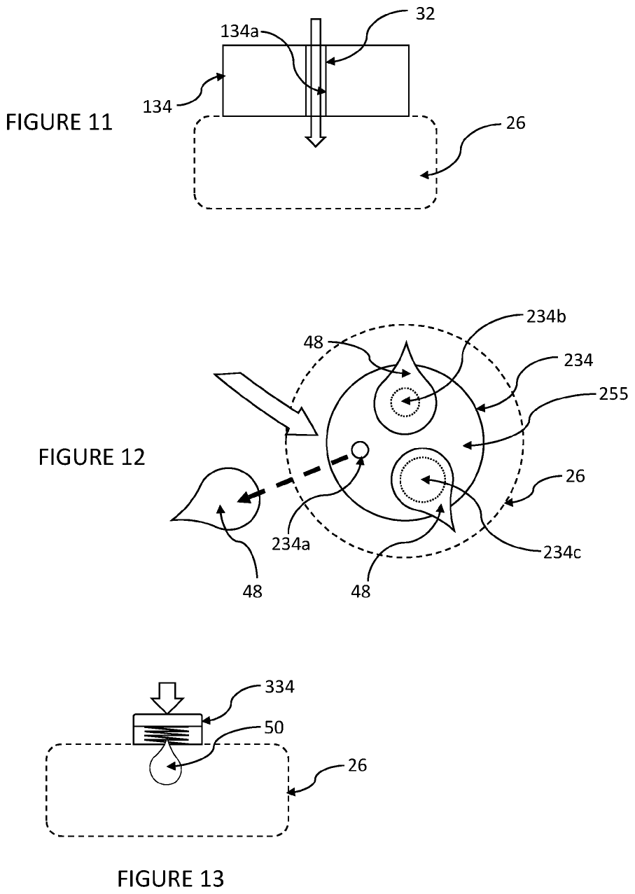

FIG. 11 illustrates representative portions of another example in which the driver 26 as previously described is associated with a regulator 134. The regulator 134 includes an orifice 134a that restricts inflow of air or other fluid into the driver 26 for pressure equalization. The size and length of the orifice 134a may be tailored to control the rate of inflow in order to control the discharge rate of the substance. In examples, the orifice 134a has a ratio of diameter to length of approximately 0.01:1 to 1:1000.

FIG. 12 illustrates representative portions of another example in which the driver 26 as previously described is associated with a regulator 234. In this example, the regulator 234 includes multiple orifices 234a/234b/234c of different sizes (cross-sectional areas) and/or shapes (e.g., tapered along length). Each orifice 234a/234b/234c is initially covered with a respective seal 48, such as a foil seal. The user may select which orifice 234a/234b/234c to unseal depending on the desired discharge rate. For example, the seal 48 of the smallest orifice 234a is removed for the highest restriction of inflow, and thus the slowest discharge rate. The seal 48 of the largest orifice 234c is removed for the lowest restriction of inflow, and thus the highest discharge rate. The seal 48 of the intermediate orifice 234b is removed for an intermediate restriction of inflow, and thus an intermediate discharge rate. As will be appreciated, the regulator 234 may include additional orifices of other sizes, to provide additional discharge rate options.

FIG. 13 illustrates representative portions of another example in which the driver 26 as previously described is associated with a regulator 334. In this example, the regulator 334 is a fixed volume pump. The fixed volume pump is manually operable (though it alternatively could be operated by electronics or other type of controller) to release a fixed volume 50 of air into the driver 26. The fixed volume pump may be operated repeatedly in order to incrementally release the pressure differential of the driver 26 and concomitantly discharge controlled increments of the substance. For example, the fixed volume pump is selectively activated to provide fixed increments of air. Each fixed increment of air causes the driver 26 to convert a corresponding increment of its potential energy to kinetic energy. The driver 26 thus expands by a corresponding increment and thereby causes an increment of the substance to be discharged. In this manner a user can precisely control the amount of substance delivered.

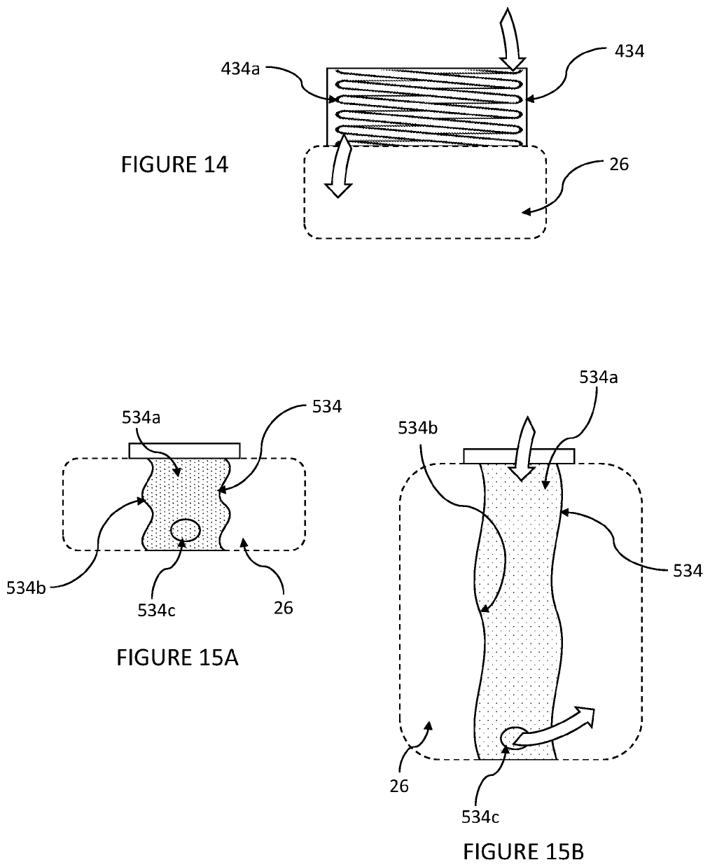

FIG. 14 illustrates representative portions of another example in which the driver 26 as previously described is associated with a regulator 434. In this example, the regulator 434 includes an elongated flow restriction tube 434a. Incoming air or other surrounding substance flowing into the driver 26 through the flow restriction tube 434a experiences a head loss (pressure loss) that restricts inflow. The restricted inflow serves to control the expansion rate of the driver 26 and thus concomitantly control the discharge rate of the substance. As an example, the tube 434a has an inner diameter from 0.001 mm to 1 mm and a length from 5 mm to 1000 mm.

FIG. 15A illustrates representative portions of another example in which the driver 26 as previously described is associated with a regulator 534. In this example, the regulator 534 includes an open cell foam 534a that is disposed in the driver 26 and that serves as the inlet port into the driver 26. The open cell foam 534a is sealed in a flexible encasement 534b in the driver 26 and has an outlet 534c that opens to the surrounding volume in the driver 26. Air or other substance flowing into the driver 26 for pressure equalization thus flows through the open cell foam 534a and through the outlet 534c into the surrounding volume of the driver 26 to release the driver 26. The open cell foam 534a is initially compressed. In the compressed state the cells of the open cell foam 534a are collapsed. As a result, the flow path through the open cell foam 534a for inflow into the driver 26 is initially restricted. As the driver 26 expands, as shown in FIG. 15A, the open cell foam 534a decompresses. The decompression opens the cells of the open cell foam 534a. As a result, as the open cell foam 534a decompresses there is less flow restriction and a greater rate of inflow through the open cell foam 534a into the driver 26. The open cell foam 534a thus modulates the inflow rate, thereby modulating the expansion rate of the driver 26 and concomitantly the discharge rate of the substance. In one example, the open cell foam 534a is made of a low-density open cell foam, such as polyurethane foam, with cell sizes in a range of 0.1 to 1.0 mm, a medium-density open cell foam, such as reticulated foam, with cell sizes in a range of 1 to 10 mm, or high-density open cell foam, such as melamine foam, with cell sizes in a range of 0.01 to 0.1 mm, or combinations of these.

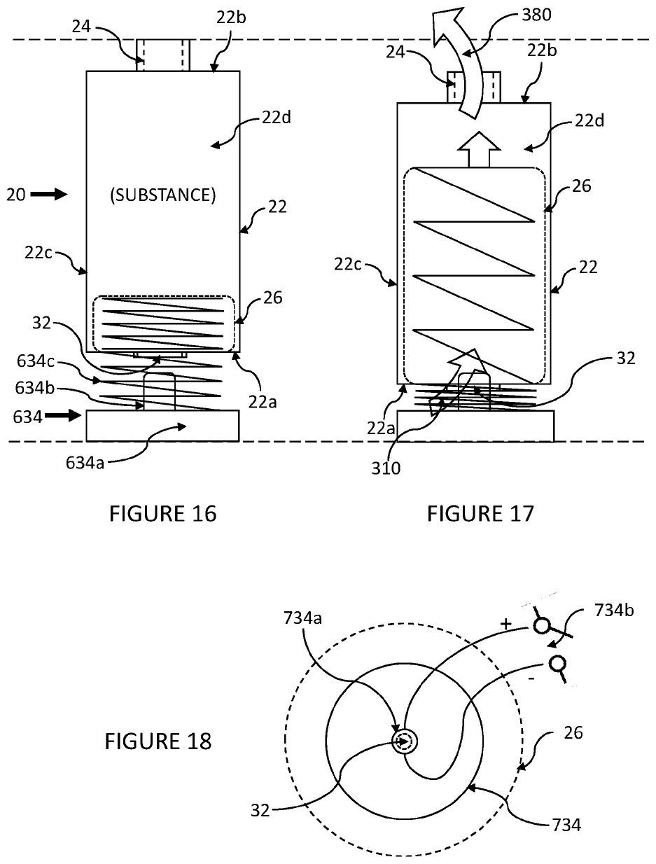

FIG. 16 illustrates representative portions of another example in which the driver 26 as previously described is associated with a regulator 634. In this example, the regulator 634 includes a base 634a, a stem 634b, and a coil spring 634c disposed on the stem 634b. The stem 634b is aligned with the inlet port 32 (which is initially sealed) of the driver 26. The coil spring 634c holds the stem 634b off of the inlet port 32. Upon depression of the base toward the container 22, as shown in FIG. 17, the coil spring 634c compresses and the stem 634b moves toward the inlet port 32. The movement of the stem 634b opens the inlet port 32, such as by piercing a seal in the inlet port 32 or moving a valve in the inlet port 32. Surrounding air is then permitted to enter the driver 26 for pressure equalization, expansion of the driver 26, and discharge of the substance from the outlet 24.

In the example shown, the pump 20 is used in a vertical position, with the substance above the driver 26. The base 634a may be depressed via a downward movement of the container 22 with the base 634a supported against a fixed surface. Such a configuration may be useful for dermatological implementation, such as a soap or cream dispenser, in which the user can simply hand-push the container 22 downwardly to dispense the substance.

FIG. 18 illustrates representative portions of another example in which the driver 26 as previously described is associated with a regulator 734. In this example, the regulator 734 includes a closure member 734a that is connected to an electric circuit 734b. The closure member 734a initially seals the inlet port 32. Upon activation of the electric circuit 734b, the closure member 734a unseals the inlet port 32 to permit inflow into the driver 26 for pressure equalization, expansion of the driver 26, and discharge of the substance from the outlet 24. In one example, the closure member 734a is a phase change material that is thermally activated by the electric circuit 734b. For instance, activation of the electric circuit 734a generates resistance heating. The heat causes the phase change material to soften or melt, thereby allowing the pressure of the inflow air to overcome the seal and flow into the inlet port 32. In this regard, the phase change material may be a wax, but is not limited thereto. In another example, the closure member 734a is a thermally degradable material. For instance, the closure member 734 is a polymer film and activation of the electric circuit 734a generates an electric arc. The arc causes thermal degradation (e.g., burning) of the polymer film, thereby opening the inlet port 32.

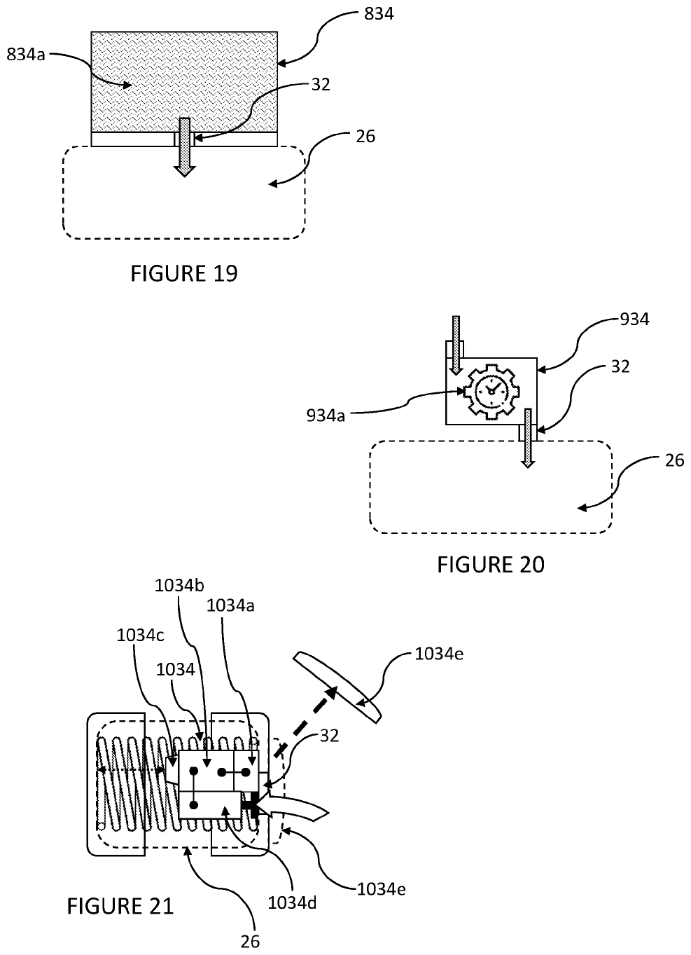

FIG. 19 illustrates representative portions of another example in which the driver 26 as previously described is associated with a regulator 834. In this example, the regulator 834 includes a viscous fluid 834a (of either Newtonian or non-Newtonian). Opening of the inlet port 32 exposes the viscous fluid 834a to the vacuum in the driver 26, thereby drawing the viscous fluid 834a into the driver 26 for pressure equalization. As a result of the high viscosity of the viscous fluid 834a, the viscous fluid 834a flows slowly into the driver 26, thereby modulating the expansion rate of the driver 26 and concomitantly the discharge rate of the substance. As the fluid is pulled through the inlet port 32, the shear will either increase or decrease the viscosity. Since the vacuum created per displacement of the driver 26 will be less as it expands (because the linear distance of the displacement creates a smaller change in volume), the type of fluid may facilitate leveling out the speed by which the substance is expelled from the container 22. As an example, the viscous fluid 834a may be, but is not limited to, water, gel, oil, or combinations thereof.

FIG. 20 illustrates representative portions of another example in which the driver 26 as previously described is associated with a regulator 934. In this example, the regulator 934 includes a mechanical clock valve 934a. The mechanical clock valve 934a is operable to periodically permit an inflow of air into the driver 26, modulating the expansion rate of the driver 26 and concomitantly the discharge rate of the substance. The clock valve 934a may include a spring-loaded diaphragm that operates on a pressure differential.

FIG. 21 illustrates representative portions of another example in which the driver 26 as previously described is associated with a regulator 1034. In this example, the regulator 1034 is an electro-mechanical controller and includes a battery 1034a, electrical circuitry 1034b including an accelerometer, a displacement sensor 1034c, and a solenoid 1034d. A cover 1034e initially covers and seals an inlet into the air-activated battery 1034a. The solenoid 1034d is operable to open and close the inlet port 32 into the driver 26. The displacement sensor 1034c detects expansion position of the driver 26 and may include vision, optical, photonic, laser, acoustic, stress/strain, capacitive or magnetic sensors. The circuitry 1034b is configured to operate the solenoid 1034d responsive to the expansion of the driver 26 and thereby modulate the rate of expansion and concomitantly the discharge rate of the substance. The circuitry 1034b may communicate through a wired or wireless connection with external electronics, which may in turn control the regulate and associated discharge rate. The circuit 1035b may determine the device orientation relative to gravity for modulating outlet flow for larger volume devices where the mass of the contents relative to gravitational orientation may affect the forces acting on the expanding driver 26.

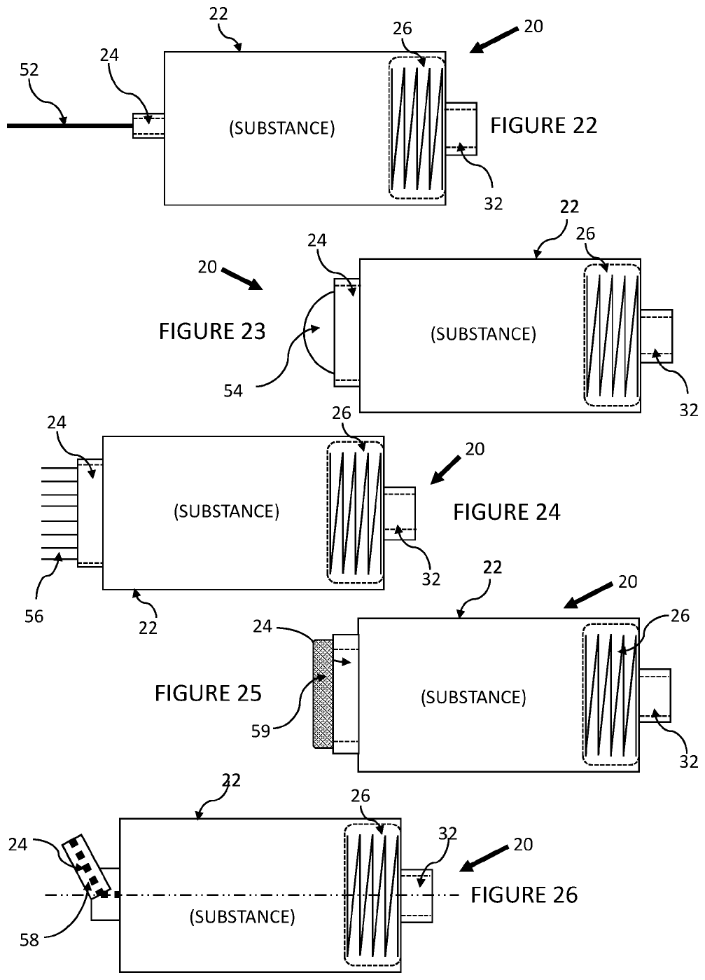

The pumps described herein may also be used in combination with various types of attachments at the outlet 24. For example, as shown in FIG. 22, the pump 20 has a hollow needle attachment 52 through which the substance is discharged for injection into a body. In FIG. 23, the pump 20 has a rollerball attachment 54 onto which the substance is discharged for distribution onto a surface. In FIG. 24, the pump 20 has a brush attachment 56 into which the substance is discharged for distribution onto a surface. In FIG. 25, the pump 20 has a foam applicator tip attachment 59 onto which the substance is discharged for distribution onto a surface.

In prior examples, the outlet 24 opens in an axial direction with respect to the central axis A of the container 22. However, as shown in FIG. 26, the outlet 24 is alternatively oriented radially such that the substance will be discharged in a non-axial direction. As will be appreciated, the geometry of the outlet 24 may be modified to other geometries that are tailored to the particular implementation of the pump 20.

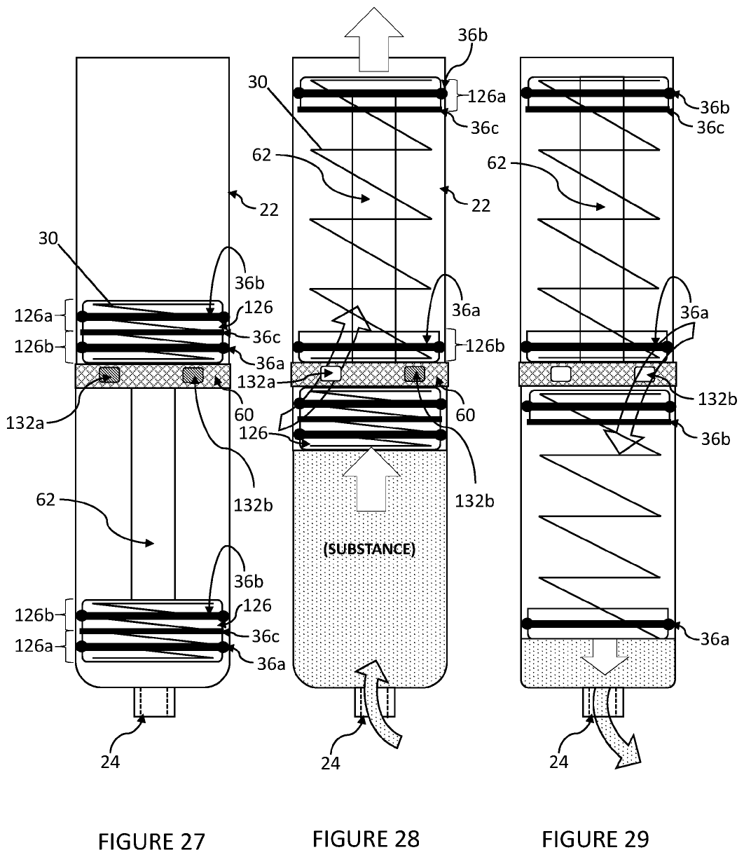

FIG. 27 illustrates another example in which there are two drives 126. The container 22 includes a partition 60 that has a first inlet 132a for a first driver 126 (top) and a second inlet 132b for a second driver 126 (bottom). A shaft 62 connects the first segment 126a of the first driver 126 on top to the second driver 126 on the bottom. As shown in FIG. 28, upon opening of the inlet 132a to release the first driver 126, the first segment 126a extends, causing the shaft 62 to move the second driver 126 in the container 22. The movement of the second driver 126 creates a vacuum in the lower portion of the container 22, drawing the substance through the outlet 24 into the container 22. As shown in FIG. 29, upon opening of the second inlet 132b to release the second driver 126, the first segment 126a extends against the substance that was drawn into container 22 and thereby discharges the substance through the outlet 24.

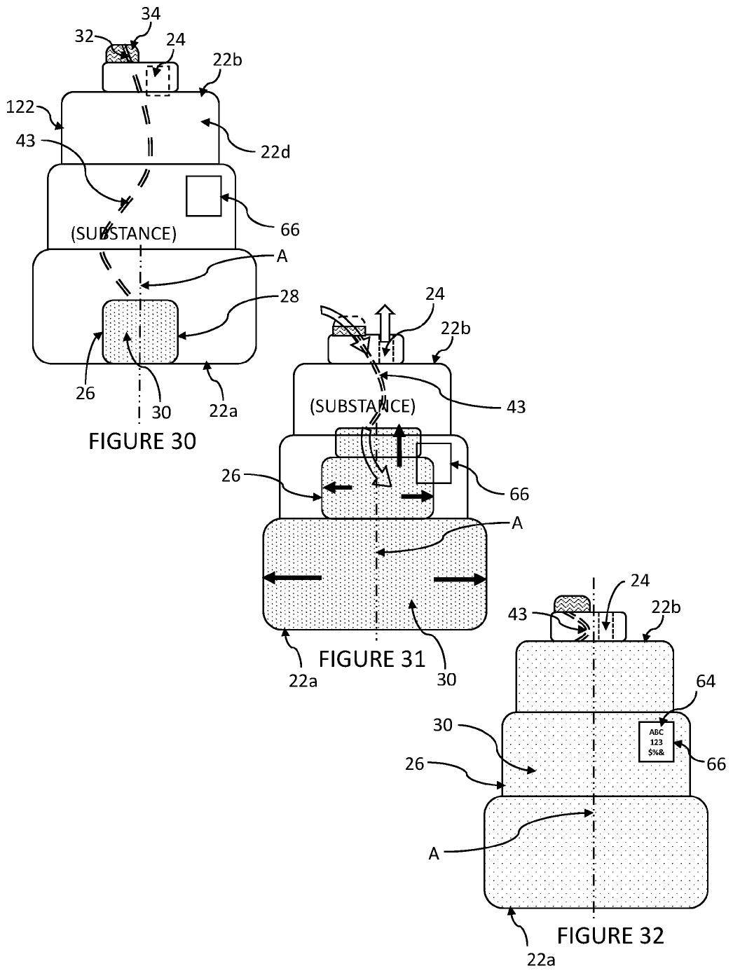

FIGS. 30, 31, and 32 illustrate another example in which the evacuated driver 26 is located inside an asymmetric container 122 with the inlet port 32 for equalizing the pressure differential. The inlet port 32 is connected via a tube 43 to the interior of the driver 26. In this example, the casing 28 of the driver is also asymmetric and, upon release, expands both axially and radially (with regard to axis A). A push valve 34 is operable by a user to open the inlet port 32 to thereby release the driver 26. Upon release, the driver expands against the substance to discharge it through the outlet 24. Release of the push valve 34 closes the inlet port 32 and thus ceases expansion of the driver 26. For example, the user depresses the push valve 34 with a finger to release the driver 26 and then may remove the finger to cease expansion/discharge. The closing of the inlet port 32 and ceasing of discharge prevents backfill of air into the container 122 by occupying the displaced interior container volume 22d, which may be of benefit for biological substances that can otherwise spoil with exposure to oxygen (air) after the container 122 is opened.

The driver 26 may include at least one marker 64 which becomes visible through a window 66 of the container 122. On release and expansion of the driver 26, the marker 64 expands from its compressed state on the surface of the driver 26. The marker 64 may include combinations of symbols, letters, numbers, bar codes, graphics, photos, and/or images for conveying information. For example, on dispensing the substance from of the container 122, the marker 64 becomes visible through the window 66 of container 122 and the user, with a smart phone scanner, may retrieve an online coupon as a reward for using and dispensing the product susbstance. In another example, the marker 64 enables the user to visualize the amount of substance dispensed or estimate the amount of substance remaining in the container. In another example, the window 66 or markings on the container surface near the window 66 includes a complementary marker. Once the marker 64 is exposed, the marker 64 and the complementary marker together form a full marker. For example, the complementary marker shows a portion of a coupon value and the marker 66 completes the value. Since the complementary marker is initially visible, the user may be enticed to use the product in order to see what the value of the coupon will be once completed by the marker 66. Such features may be useful for consumer substances, such as detergents or cosmetics. Moreover, the driver 26 in this case serves as an anti-theft feature, as the coupon is not visible/useable until the driver 26 is released and the coupon cannot, therefore, be stolen without using the device.

As indicated, the end-uses of the pumps disclosed herein are not limited. One example useful implementation is in dermatology or pharmaceutical field, where it may be necessary to keep the substance and the drive separate to prevent oxygen from degrading the substance (e.g., a dermatology drug) or to prevent oxygen (air) from contributing to bacteria/fungal growth in the substance. The pump also enables long term storage of the potential energy before use. Additionally, by eliminating a battery and a motor from a drug delivery device, the amount of landfill e-waste is reduced. Furthermore, the use of the pressure differential enables devices that use the pump to be simplified with lower mechanical complexity and, in some cases, reduce the stress on key parts of the devices. For example, instead of a mechanical latch with high, focused forces the pump holds the energy under a distributed pressure load and releases the energy by release/breaking the vacuum.

An example method for use of any of the pumps herein in the medical field includes connecting the pump to a patient for delivery of a medicament, releasing the pressure differential via the pressure equalization port to cause the potential energy in the spring device to convert to kinetic energy such that the evacuated driver expands against the medicament in the interior volume and thereby discharges the medicament through the at least one outlet, the expansion drawing an inflow into the evacuated driver through the pressure equalization port, and after delivery of the medicament to the patient, disconnecting the pump from the patient. As indicated, the disclosed pumps are not limited to the medical field and may also be used for delivery of other types of substances such as but not limited to, cosmetics, condiments, soaps, cleaning solutions, oils, or mixtures of these and/or other substances. For instance, the pump with the substance in it is brought into proximity of a point of delivery and the pressure differential is released in order to discharge the substance toward the point of delivery.

The configurations disclosed herein also facilitate maximizing the use of the container contents. The expansion of the driver wipes the container walls and pushes the lowest contents towards the dispensing point, which existing squeeze containers, hand pumps, or shake containers (i.e., the ketchup bottle dilemma) do not do. As a result, there may be advantages in food utilization (i.e., maximize food use by reducing wasted food) and reduction in the need for plastic bottles. It also reduces industrial waste in the production and consumption of cosmetics, detergents, soaps, and medications by maximizing use at the point of dispensing. Another advantage lay in reducing skin contact with a container substance. For example, in existing cosmetic containers skin bacteria can ruin expensive contents when the user must scoop the cosmetic from a container with fingers. The use of the evacuated drive to push the contents to the user without skin contact enables better preservation of the cosmetic for long-term preservation between applications, which may be especially valuable for specialty cosmetics with elevated costs for small volumes of substance.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A pump comprising:
a container having an interior volume for holding a substance;
at least one outlet associated with the interior volume for discharging the substance; and
a driver disposed in the interior volume, the driver having a stored potential energy that is releasable as kinetic energy and, upon release, the driver being expandable against the substance in the interior volume to thereby discharge the substance through the at least one outlet.

2. The pump as recited in claim 1, wherein the driver includes a casing and a spring device in the casing, the casing being evacuated to define a pressure differential that stresses the spring device to provide the stored potential energy.

3. The pump as recited in claim 2, wherein the driver is configured to release the stored potential energy by release of the pressure differential.

4. The pump as recited in claim 3, wherein the driver has a pressure equalization port that is operable to release the pressure differential and, upon release, the spring device expanding to increase the volume of the casing and thereby generate a secondary vacuum in the casing.

5. The pump as recited in claim 4, wherein the container includes first and second ends, the at least one outlet is in the first end, and the pressure equalization port is in the first end.

6. The pump as recited in claim 1, wherein the driver includes first and second segments, the first segment includes a first seal that seals against an interior surface of the container and the second segment includes a second seal that seals against an interior surface of the container, the first and second seals isolating a region from the remainder of the interior volume.

7. The pump as recited in claim 6, wherein the driver includes a spring device in the region, the region is evacuated to define a pressure differential that stresses the spring device to provide the stored potential energy, the first segment is translatable in the container, the second segment is fixed in the container, and upon release of the pressure differential the spring device moves the second segment against the substance to discharge the substance through the at least one outlet.

8. The pump as recited in claim 7, further comprising a conduit in the container and extending through the driver, the conduit connecting the interior volume with the at least one outlet for discharging the substance through the conduit.

9. The pump as recited in claim 8, further comprising a passive valve associated with the conduit, the passive valve held closed by the pressure differential and opening upon release of the pressure differential to permit the substance to flow into the conduit.

10. The pump as recited in claim 1, wherein the at least one outlet includes an attachment selected from the group consisting of a needle, a roller ball, a brush, a foam applicator, a directional nozzle, and combinations thereof.

11. The pump as recited in claim 1, further comprising a stem that is attached with the driver, the stem being moveable to retract the driver in the container and thereby draw the substance into the interior volume.

12. The pump as recited in claim 1, wherein the driver occupies a region in the container, and the region is fluidly isolated from the interior volume.

13. The pump as recited in claim 1, further comprising a thermal material in the driver, the thermal material being activatable responsive to release of the driver to change the temperature of the substance.

14. The pump as recited in claim 1, wherein the container includes a window and the driver includes a marker, the marker becoming visible through the window upon release of the driver.

15. A pump comprising:
a container having an interior volume;
a substance in the interior volume;
at least one outlet associated with the interior volume for discharging the substance; and
at least one evacuated driver disposed in the interior volume, the evacuated driver defining a pressure differential that stresses a spring device of the evacuated driver to store a potential energy, the evacuated driver having a pressure equalization port that is operable to release the pressure differential and, upon release, to cause the potential energy in the spring device to convert to kinetic energy such that the evacuated driver expands against the substance in the interior volume and thereby discharge the substance through the at least one outlet.

16. The pump as recited in claim 15, wherein the pressure equalization port includes a regulator that restricts inflow into the evacuated driver in proportion to expansion of the evacuated driver.

17. The pump as recited in claim 16, wherein the regulator includes at least one passive flow restrictor selected from an orifice, a flow restriction tube, an open cell foam, a viscous fluid, and combinations thereof.

18. The pump as recited in claim 16, wherein the regulator includes at least one active flow control selected from a valve, an electrical element, a solenoid, a phase change material, a thermally degradable material, a fixed volume pump, and combinations thereof.

19. The pump as recited in claim 18, wherein the regulator includes the fixed volume pump, the fixed volume pump being operable to release a fixed volume of air into the evacuated driver to incrementally release the pressure differential.

20. The pump as recited in claim 15, wherein the at least one evacuated driver includes two evacuated drivers, and each of the two evacuated drivers is segmented into a moveable first segment, a fixed second segment, and the two evacuated drivers are interconnected by a shaft.

21. A method for controlling discharge from a pump, the method comprising:
providing a pump that includes a container that has an interior volume, a medicament in the interior volume, at least one outlet associated with the interior volume for discharging the medicament, and at least one evacuated driver disposed in the interior volume, the evacuated driver defines a pressure differential that stresses a spring device of the evacuated driver to store a potential energy, the evacuated driver has a pressure equalization port;
connecting the pump to a patient for delivery of the medicament;
releasing the pressure differential via the pressure equalization port to cause the potential energy in the spring device to convert to kinetic energy such that the evacuated driver expands against the medicament in the interior volume and thereby discharges the medicament through the at least one outlet, the expansion drawing an inflow into the evacuated driver through the pressure equalization port; and
after delivery of the medicament to the patient, disconnecting the pump from the patient.
